(19) United States
(12) Patent Application Publication
(10) Pub. No.: US 2008/0284167 A1
(43) Pub. Date: Nov. 20, 2008

(54) LOW-VOLUME FITTINGS

(76) Inventors: Eugene Lim, Lafayette, CA (US); Richard Keenan, Livermore, CA (US); Michael Butler, Dublin, CA (US)

Correspondence Address: STEVEN VOSEN, 1563 SOLANO AVENUE #206, BERKELEY, CA, 94707, US

(21) Appl. No.: 12/122,009

(22) Filed: May 16, 2008

Related U.S. Application Data
(60) Provisional application No. 60/939,012, filed on May 18, 2007.

(51) Int. Cl. F16L 25/00

(52) U.S. Cl. 285/382

(57) ABSTRACT

Fitting that is compatible with standard fittings is described. In one embodiment, the end of one of a matched set of fittings is provided with an elastomeric material. The elastomeric material deforms when the matched fittings are coupled, resulting in a reduced volume. In certain embodiments, a tube is provided through the tube which is attached at a proximal end and extends to the distal end of the elastomeric material. When the fitting are coupled the tube protrudes past the end of the elastomeric material and into the coupled fitting. In certain embodiments the fitting otherwise conforms to International Standard ISO 594.

### CROSS-REFERENCE TO RELATED APPLICATIONS

This application claims the benefit of U.S. Provisional Application No. 60/939,012, filed May 18, 2007, the entirety of which is incorporated herein by reference.

### BACKGROUND OF THE INVENTION

1. Field of the Invention

The present invention generally relates to fittings for connecting tubing, and more particularly to connectors that have small excess internal volumes.

2. Discussion of the Background

Medical and instrument tubing is typically joined by providing a connector that includes matching fittings on the ends of tubes to be joined. One common connector includes conical fittings. Conical fittings include a matched set of male and female fittings each having conical surfaces that, when the fittings are connected, provides leak-proof connections between tubes. Conical fittings that form a seal by forcing the fittings together are sometimes referred to as “slip fittings.” Conical fittings that also include threaded elements to secure the fittings are sometimes referred to as “lock fittings.”

Standards have been developed to permit compatibility of standard fittings. An example of specifications for conical fittings used for medical applications may be found, for example, in the International Standard ISO 594 titled “Conical fittings with 6% (Luer) Taper for syringes, needles and certain other medical equipment,” and referred to herein as the “ISO Luer Standard.” Fittings meeting the ISO Luer Standard are referred to herein as “Luer fittings.”

To facilitated an understanding of embodiments of the present invention, FIGS. 1 through 3 are views of a prior art Luer slip fitting, where FIG. 1 a side view of a male Luer fitting 10 and a matching female Luer fitting 20, and FIG. 2 is a sectional view 2-2 of the fittings. Male Luer fitting 10 has a distal end 12 for inserting into female Luer fitting 20 and a proximal end 14 for attaching to a tube (shown in FIG. 3). Female Luer fitting 20 has a distal end 22 for accepting male Luer fitting 10 and a proximal end 24 for attaching to a tube (shown in FIG. 3). Proximal ends 14 and 24 may each include, for example, a barb 13 to accept the end of a tube.

The following discussion relates to fittings 10 and 20 that conform to the ISO Luer Standard. Fittings 10 and 20 each include surfaces having the shape of a conical frustum of a given slope, resulting in a conical angle θ, as shown. More specifically, male fitting 10 has an external frustoconical surface 11 with having a height A from a proximal base 17 to a distal top 16 having a diameter B. Female fitting 20 includes an internal frustoconical surface 21 that matches at least a portion of surface 11. Specifically, surface 21 has a depth D extending from an opening at the distal base 27 with a diameter C to a proximal top 26. The height A, diameters B and C and depth D, are mandated by the ISO Luer Standard, and define contacting surfaces. While the shape of contacting surfaces of surfaces 11 and 21 are particularly important for portions where the male and female fittings touch, the shape over other portions may have different conical angles or shapes, particularly at more proximal portions of the male and female fittings (compare, for example, FIG. 1 with FIGS. 4 and 5 of the first edition of the ISO Luer Standard).

Neither the cross-sectional size nor cross-sectional shape of passageways 15 and 25 are specified in the ISO Luer Standards. For illustrative purposes, fittings 10 and 20 are shown having passageways 15 and 25, respectively, that may each have approximately constant cross-sectional areas in a flow direction. Passageway 15 has a distal end 19 at top 16 and passageway 25 has a distal end 29 at top 26.

While standards, such as the ISO Luer Standard, provide a framework for producing interchangeable connectors, the volume and shape of the volume varies from fitting to fitting. This variation may present problems for low flow or low volume systems, or for systems requiring that the fluid within the connectors be exposed to only smooth surfaces.

FIG. 3 is a sectional view 2-2 where fittings 10 and 20 are connected and a portion of surfaces 11 and 21 are in contact, where the portion is indicated by the length X. The ISO Luer Standard mandates that tops 16 and 26 be separated by a distance, which is indicated as distance between distal ends 19 and 29 as E=X−D. The value of E is not specified by the ISO Luer Standard, but is required to be greater than zero, resulting in a “dead space” within connected fittings 10 and 20. The “dead space,” as used herein, refers to volume within coupled fittings 10 and 20 that is: 1) not within passageways 15 or 25; and 2) is not sealed off from passageways 15 or 25. In other words, the dead space is any volume outside of the passageways leading into the coupled fittings into which fluid may migrate. Thus, for example, the dead space in the fittings of FIG. 3 is a volume 300 that is between distal end 19 of passageway 15 and distal end 29 of passageway 25. Because the distance E is non-zero, there is a finite volume V of dead space 300 within coupled fittings 10 and 20.

It is understood that, in some prior art embodiments, one or more of passageway 15 or 25 may terminate at top 16 or 26, respectively, with a beveled or some other shaped surface, and thus the shape of the dead space may have some other shape.

In the simplified illustration of FIG. 3, the frustoconical dead space 300 is approximately cylindrical, with a diameter of approximately B and height E, resulting in V=E*πB²/4. Since the value of E, as well as the cross-sectional diameter of passageways 15 and 25, are not specified in the ISO Luer Standard, dead space volume V will not necessarily be the same from one fitting to another. For ISO Luer Standard compliant fittings, the minimum height E is approximately 1.75 mm and B is approximately 3.9 mm, for a volume V of approximately 21 μl. This calculation of V is approximate and for illustrative purposes, as there are no ISO Luer Standards for the internal size or shape of Luer fittings.

The variation in dead space volume and shape from fitting-to-fitting within the ISO Luer Standard may present a problem for low volume or low flow systems. In addition, dead space is believed to be a problem in lines that are used to provide patients with medication. See, for example, the letter to the CANADIAN JOURNAL OF ANAESTHESIA, http://www.cja-jca.org/cgi/reprint/42/7/658.pdf). Thus, for example, if a line with such a connector is used to provide a patient with medication, some of the medication may remain trapped in the dead space. If the line is later flushed with saline, unknown and potentially dangerous amounts of the medication may be flushed from the dead space and infused into the patient.

For all of these reasons there is need for connectors having predictable, and preferably low, internal volumes.

### BRIEF SUMMARY OF THE INVENTION

The present invention overcomes the limitations and problems of the prior art by providing a fitting that reduces the volume of standard fittings.

In certain embodiments, a male fitting is provided. The male fitting extends longitudinally from proximal end to a distal end, and includes a first portion for mating with a matched female fitting; and a second portion having a length from the first portion to the distal end, where the length is greater than approximately 1.75 mm.

In certain other embodiments, a male fitting is provided. The male fitting extends longitudinally from proximal end to a distal end, and includes a first portion for mating with a matched female fitting, and a second portion between the first portion and the distal end, where the second portion includes an elastomeric material.

In certain embodiments, a male fitting conforming to International Standard ISO 594 is provided. The male fitting is connectable to a female fitting conforming to International Standard ISO 594. When the male fitting connected to the female fitting has a dead space, where the dead space has a volume of less than 15 μl.

In certain embodiments, the dead space has a volume that is: less than 15 μl; less than 10 μl; less than 5 μl; less than 2 μl; less than 1 μl; or is approximately zero.

In certain embodiment, the extension has a length of: approximately 1.75 mm; approximately 6 mm; greater than 1 mm; greater than 2 mm; greater than 3 mm; greater than 4 mm; greater than 5 mm; greater than 7 mm; greater than 8 mm; or greater than 9 mm. Alternatively, the extension have a length within the following ranges: between 1 mm and 10 mm; between 2 mm and 9 mm; between 3 mm and 8 mm; or between 4 mm and 7 mm.

These features together with the various ancillary provisions and features which will become apparent to those skilled in the art from the following detailed description, are attained by the fitting of the present invention, preferred embodiments thereof being shown with reference to the accompanying drawings, by way of example only, wherein:

### BRIEF DESCRIPTION OF THE SEVERAL VIEWS OF THE DRAWING

FIG. 1 a side view of a prior art male Luer slip and a matching female Luer fitting;

FIG. 2 is a sectional view 2-2 of the prior art fittings of FIG. 1;

FIG. 3 is a sectional view 2-2 where fittings are fully connected;

FIG. 4 is a sectional view of one embodiment of a male Luer fitting;

FIG. 5 is a sectional view of the male Luer fitting of FIG. 4 mated with a female Luer fitting;

FIG. 6 is a perspective view of an embodiment of a male Luer lock connector and a matching female Luer lock connector;

FIG. 7 is a perspective view of the male Luer fitting of FIG. 6;

FIG. 8 is a sectional view 8-8 and FIG. 9 is a sectional view 9-9 of the male Luer fitting of FIG. 6;

FIG. 10 is a sectional view 8-8 of the fitting of FIG. 8 as attached to a tube;

FIG. 11 is a detailed sectional view 11-11 of the female Luer connector of FIG. 6;

FIG. 12 is a sectional view 8-8 and 11-11 showing the male fitting and female fitting of FIG. 6 connected; and

FIG. 13 is a detailed sectional view 13-13 of FIG. 12.

Reference symbols are used in the Figures to indicate certain components, aspects or features shown therein, with reference symbols common to more than one Figure indicating like components, aspects or features shown therein.

### DETAILED DESCRIPTION OF THE INVENTION

Embodiments will now be described with reference to Figures of conical Luer fittings. It is understood that the features described herein may be used in other fittings to reduce or otherwise modify the internal volume of connectors, as described herein. In certain embodiments, the coupled fitting presents a nearly uniform flow diameter for fluids within the fitting.

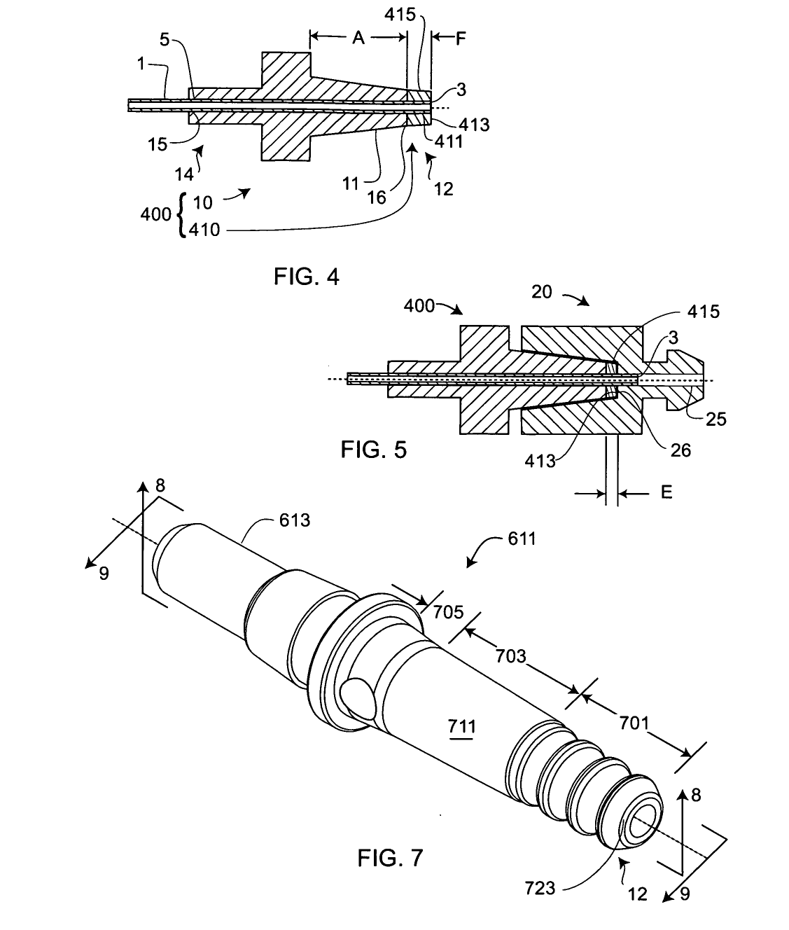

FIG. 4 is a sectional view of one embodiment of a male Luer fitting 400, which may be generally similar to fitting 10, except as further detailed below.

In one embodiment, fitting 400 includes a fitting 10 and also includes an extension 410 at distal end 12. Extension 410 has a passageway 411 and an outer surface 415, and that protrudes away from fitting 10 by a distance F away from top 16 to a distal surface 413. In one embodiment, fitting 10 includes a rigid or semi-rigid material. Thus, for example and without limitation, fitting 10 includes a metal or a plastic. Examples of plastics for fitting 10 include, but are not limited to, a rigid thermoplastic such as polycarbonate (PC) radiation stable PC, acrylonitrile butadiene styrene (ABS), poly(methyl methacrylate) (PMMA), polyvinyl chloride (PVC), or polypropylene (PP). In another embodiment, extension 410 includes a material that is rigid or semi rigid. In yet another embodiment, extension 410 includes a material that is more compliant that that of fitting 10, and is preferably resiliently deformable. Examples of materials for extension 410 include, but are not limited to elastomeric materials, such as a thermoplastic elastomer (TPE). Alternate materials for the extension include thermoplastic urethane (TPU) or silicone.

In one embodiment, fitting 400 includes a passageway 15 and extension 410 includes passageway 411 that can accept a tube 1. Specifically, an accepted tube 1 passes through passageways 15 and 411, with a tube end 3 flush with distal surface 413. In one embodiment, glue is added to proximal location 5, such that tube 1 is fixed to the proximal end of the fitting. Thus, if extension 410 is compressed tube end 3 will protrude distally beyond distal surface 413. In another embodiment, tube end 3 is within one of passageways 15 or 411.

FIG. 5 is a sectional view of the fitting 400 coupled to female Luer fitting 20. The coupled connectors are shown for illustrative purposes, and do not limit the scope of claims of fitting 400. Extension 410, when located within coupled fittings 400 and 20 may reduce the volume of the dead space in one or more of several ways. First, the presence of extension 410 within coupled fittings 400 and 20, whether the extension is formed from an elastomeric, rigid, or semi-rigid material, reduces the volume of dead space from that of prior art fittings, as in FIG. 3. In one embodiment, extension 410 is sized and is of a material resilient enough to reduce or otherwise modify the volume of connectors comprising the range of sizes permitted under the ISO Luer standards.

Second, extension 410 may seal against the inner surfaces of fitting 20, such as surface 21 or top 26, further reducing the volume into which fluid from passageways 15 or 16 may migrate.

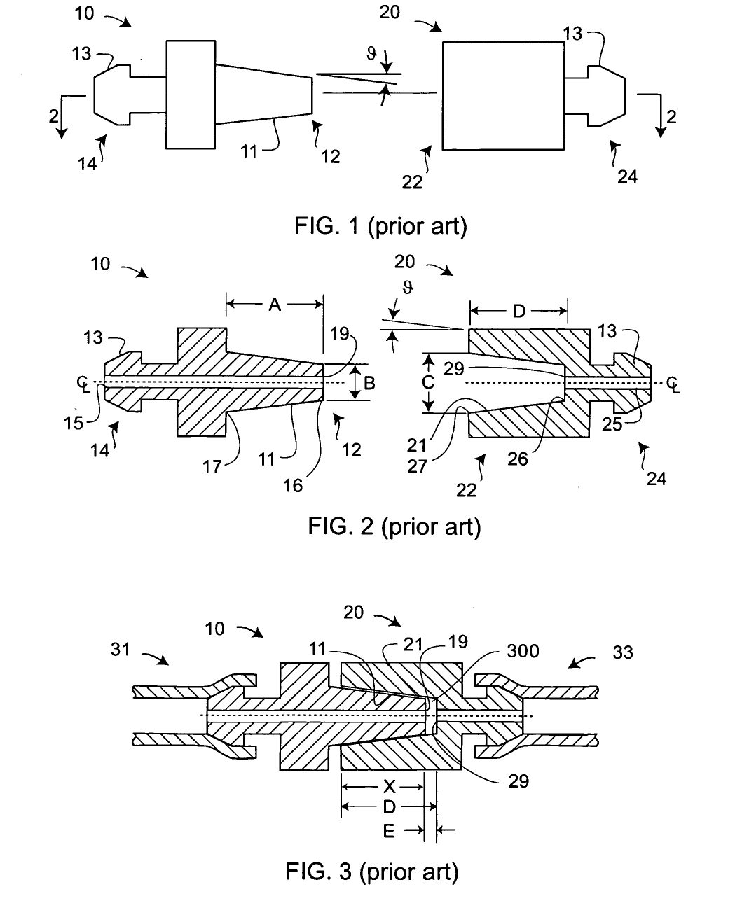

Third, tube 1 may protrude to or into fitting 20. As shown in FIGS. 4 and 5, in one embodiment extension 410 includes an elastomeric material and the distance F is equal to or greater than the distance E—that is, extension 410 is longer than the length of dead space 300 of the prior art fittings of FIGS. 1-3. When fitting 400 is used in place of fitting 10, for example, extension 410 is compressed from a length F (as in FIG. 4) to a length E, and tube end 3 extends to passageway 25 of female Luer fitting 20, reducing the dead volume to essentially zero. In addition to greatly reducing or eliminating dead space, the smooth transition of flow diameters results in smoother flow through the coupled fittings 400 and 20.

In one embodiment, the dead volume in coupled fittings 400 and 20 is less than 20 μl. In alternative embodiments, the dead volume in coupled fittings 400 and 20 is less than 15 μl, is less than 10 μl, is less than 5 μl, is less than 2 μl, or is less than 1 μl. In another alternative embodiment, the dead volume in coupled fittings 400 and 20 is approximately zero.

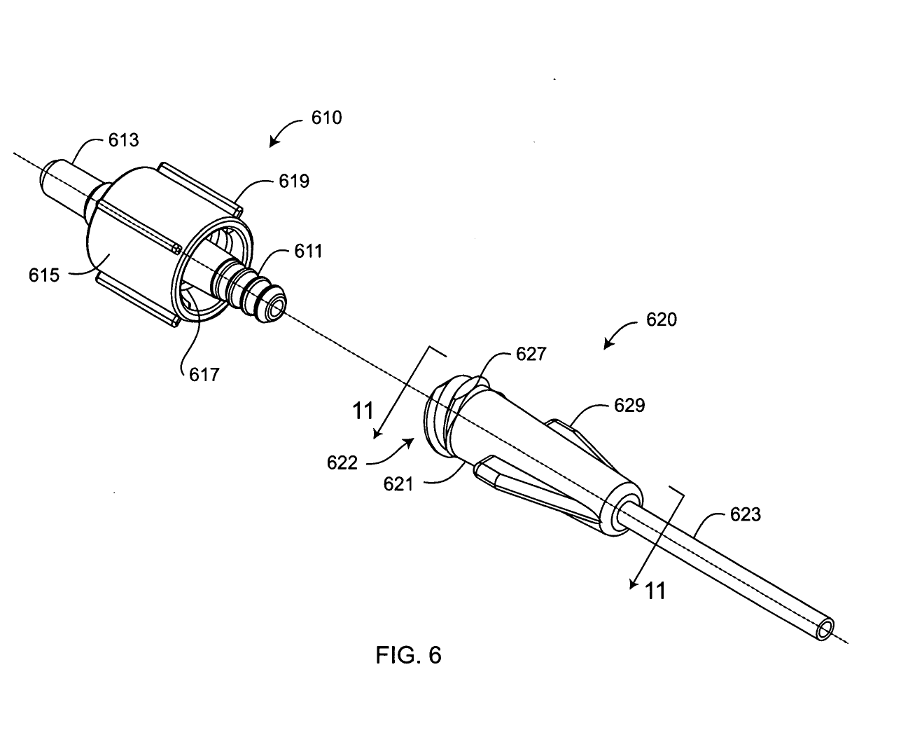

FIG. 6 is a perspective view of an embodiment of a male Luer lock connector 610 and a matching female Luer connector 620, which may be generally similar to fittings 400 and 20, except as further detailed below.

Connector 610 is a lock fitting that includes a male Luer fitting 611 having a distal end 12, and a surrounding lock element 615 having internal threads 617 and features 619 to facilitate handling the fitting. As described subsequently, fitting 610 is connectable to a tube.

Connector 620 is a matching lock fitting for connector 610. Connector 620 has a female Luer fitting 621 having an opening 622 to accept distal end 12, and external threads 627 and features 629 for facilitating handling the fitting. Threads 617 and 627 are matching threads. Connector 620 also includes a tube 623 or an element or mechanism to permit the attaching of a tube to coupling end 621. In one embodiment, fitting 621 is a prior art fitting 20.

In one embodiment, Connector 620 includes a standard locking conical fitting that conforms to a standard which may be, for example, the ISO Luer Standard. In another embodiment, connector 610 conforms to a conical fitting standard for a locking fitting, that is, it can mate with any female fitting conforming to the same standard, and also includes features and/or elements that reduce or otherwise modify the volume internal to connected fittings 610 and 620.

FIG. 7 is a perspective view of fitting 611—that is, connector 620 with lock element 615 removed, Fitting 611 includes an extension 701 at a distal location having a distal end 723, a frustoconical portion 703 including a surface 711, and a proximal portion 705. Surface 711 may be generally similar to surface 11—that is, it may mate with a matching internal frustoconical surface of a female fitting. Extension 701 may be generally similar to extension 410 in that it extends laterally beyond the distal end of frustoconical portion 703.

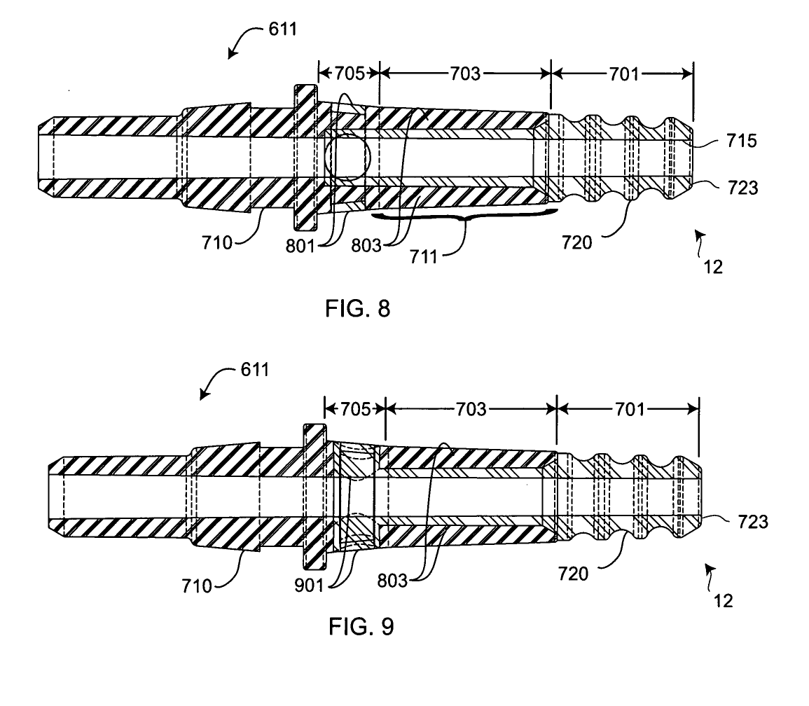

Fitting 611 is shown in greater detail in FIG. 8, which is a longitudinal sectional view 8-8 of FIG. 7 and in FIG. 9, which is an orthogonal longitudinal section view 9-9 of FIG. 7. As shown in FIGS. 8 and 9, fitting 611 has a passageway 715 and may be formed from a first material 710 and a second material 720. Material 710 is structural and includes the majority of fitting 611. Thus, for example, material 710 includes proximal end 613 and at least part of frustoconical portion 703. In one embodiment, material 710 is a MAKROLON® RX2530-1118 Polycarbonate (Bayer MaterialScience, Leverkusen, Germany).

Material 720 may form most of extension 710 and has a portion 803 that protrudes under surface 711 and forms at least part of proximal portion 705. As shown in FIG. 8, an annulus 801 of material 720 is connected via two radial portions 901 to extension 701. In one embodiment. Annulus 801 functions as an anchor for extension 710 so that the extension is not easily pulled from fitting 611. Material 720 may be, for example and without limitation, the material of extension 410. In one embodiment, the first material extension 720 is a G2706 TPE (GSL) (Kraton Polymers, Houston, Tex.). In one embodiment the longitudinal extent of extension 710 is approximately 6 mm. In alternative embodiments, the longitudinal extent of extension 710 is greater than 1 mm, is approximately 1.75 mm, is greater than approximately 1.75 mm, is greater than 2 mm, is greater than 3 mm, is greater than 4 mm, is greater than 5 mm, is greater than 7 mm, is greater than 8 mm, or is greater than 9 mm. In yet other embodiments, the longitudinal extent of extension 710 is between 1 mm and 10 mm, is between 2 mm and 9 mm, is between 3 mm and 8 mm, or is between 4 mm and 7 mm.

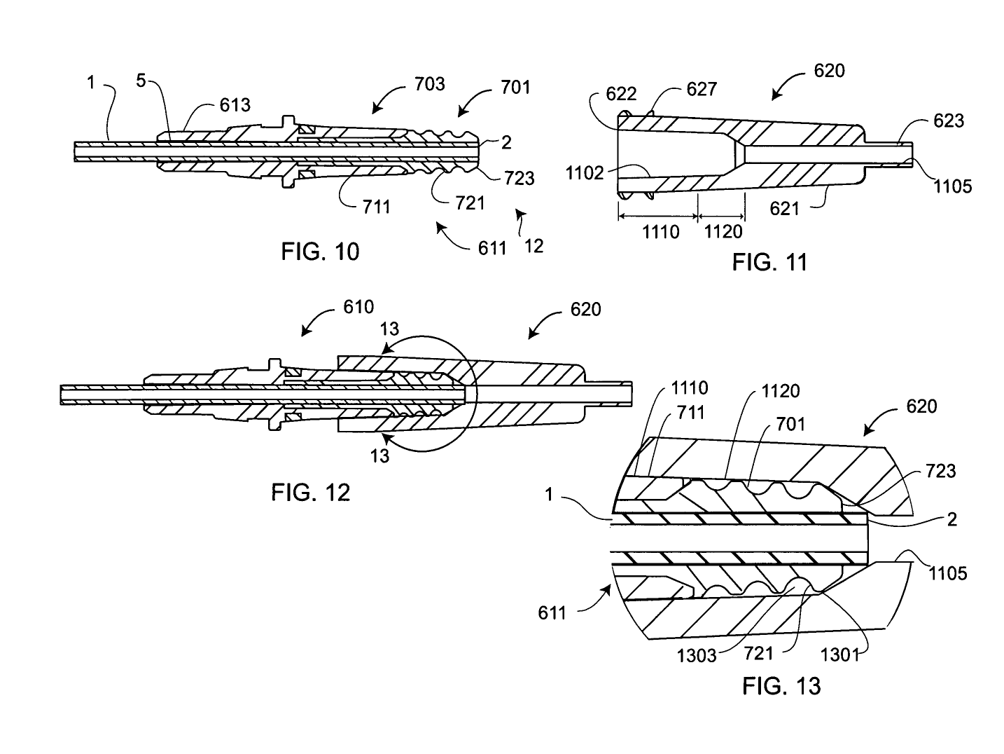

FIG. 10 is a sectional view 8-8 of the male Luer lock fitting 611 having a tube 1. Fitting 611 and tube 1 are generally similar to fitting 400 and tube 1 of FIGS. 4 and 5, except as further detailed below.

In one embodiment, tube 1 is a PVC tube. In another embodiment, tube 1 is a co-extruded tubing having a polyethylene “sleeve” inside a PVC outer tube. As shown in FIG. 10, fitting 611 is attached to tube 1 with end 3 that is approximately even with distal end 723.

In one embodiment, tube 1 is a small bore tube. Thus in one example, which is not meant to limit the scope of the present invention, the outer diameter of tube 1 is slightly less than the diameter of passageway 715. In one embodiment, the outer diameter of tube 1 is 0.060 inches and the inner diameter of the tube is 0.030 inches. In one embodiment, the outer surface of tube 1 and an inner surface of proximal end 613 are affixed at location 5 using glue, such as a UV cured glue.

Extension 701 includes an outer surface 721 having ridges. This shape provides space for extension 701 to compress into when joined with a mating fitting, as in FIG. 12 and 13, below. Alternatively, outer surface 721 may form a fluid-tight seal against fitting 620.

In one embodiment, fitting 611 is formed by injection molding material 710, which includes radial portions 901 and annulus 801. Material 720 is then overmolded or co-molded over material 710 to form extension 720 and fill in annulus 801, and radial portion 910. Tube end 3 is then inserted through proximal end 613 to distal end 723, with glue being applied location 5.

FIG. 11 is a sectional view 11-11 of the female Luer fitting 620 of FIG. 6. Fitting 620 includes a body 621 having an inner surface 1102 including a frustoconical surface 1110, an extension portion 1120, and an inner surface 1105 along a portion of the body. Portion 110 may be, for example and without limitation, a conical frustum portion compliant with the ISO Luer Standard for a female fitting, and may be a mating surface to surface 711.

FIG. 12 is a sectional view of coupled fittings 611 and 620, that is, when surfaces 702 and 1110 are in contact, and FIG. 13 is a detailed sectional view 13-13 of FIG. 12.

As shown in FIG. 12, extension 701 is forced by extension portion 1120 into a compressed configuration. Thus, for example, extension 701 may compress by up to one half its undeformed length. Since tube 1 is affixed to proximal end 613 at location 5, the distal end 3 of the tube extends beyond distal end 723 of extension 701. In one embodiment, tube end 3 extends to the most proximal end of portion 820. As a result, flow within tube 1 makes a smooth transition as it flows through fittings 610 and 620.

Also shown in FIG. 13 are details of an embodiment where extension 701 seats on the surface of extension portion 1120. Specifically, ridges of outer surface 721 are forced on to the surface of extension portion 1120 to form a fluid-tight seal, as indicated at a sealing location 1301. The seal prevents fluid from moving into the fitting. Thus, for example, there is a void 1303 between the surface and inner surface 1120 that is sealed at sealing location 1301, preventing fluid from moving from tube 1 into the void. The seal at sealing location 1301 thus prevents void 1301 from being included as part of the dead space of coupled fittings 611 and 620.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment. Furthermore, the particular features, structures or characteristics may be combined in any suitable manner, as would be apparent to one of ordinary skill in the art from this disclosure, in one or more embodiments.

Similarly, it should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment.

In summary, embodiments of fittings include several features, some or all of which may be present in any specific embodiment. In general, fittings of the present invention include one or more of the following elements: 1) an extension to a standard fitting, where the material of the extension may or may not be an elastomeric material; and/or 2) a tube extending through a standard fitting, where the tube may or may not extend to the distal end of the fitting. While embodiments of fittings are shown in various configurations relative to the coupled fitting, the present invention is not limited to specific details of how the fitting coupled to the other fitting, except as explicitly claimed. Thus, for example, in various embodiments, the claimed fitting of the coupled fittings includes a standard fitting with 1) an extension that is or is not compressed; 2) a tube having a distal end that terminates before, at, or within the coupled fitting.

Thus, the claims following the Detailed Description are hereby expressly incorporated into this Detailed Description, with each claim standing on its own as a separate embodiment of this invention.

We claim:

1. A male fitting that extends longitudinally from proximal end to a distal end, said male fitting comprising:
a first portion for mating with a matched female fitting; and
a second portion having a length from said first portion to said distal end, where said length is greater than approximately 1.75 mm.

2. The male fitting of claim 1, where said second portion includes an elastomeric material.

3. The male fitting of claim 1, where said male fitting conforms to International Standard ISO 594.

4. The male fitting of claim 1, where said male fitting further includes a tube extending from the proximal end to said distal end, where said tube is affixed to said male fitting near at or near said proximal end.

5. The male fitting of claim 1, where said first portion includes a rigid thermoplastic or a metal.

6. The male fitting of claim 1, where said length is approximately 6 mm.

7. The male fitting of claim 1, where said male fitting, when coupled to a female fitting that conforms to International Standard ISO 594, has a dead space with a volume of less than 15 μl.

8. The male fitting of claim 1, where said male fitting further includes a tube extending from the proximal end to said distal end, where said tube is affixed to said male fitting near at or near said proximal end.

9. A male fitting that extends longitudinally from proximal end to a distal end, said male fitting comprising:
a first portion for mating with a matched female fitting; and
a second portion between said first portion and said distal end, where said second portion includes an elastomeric material.

10. The male fitting of claim 9, where said male fitting conforms to International Standard ISO 594.

11. The male fitting of claim 9, where said male fitting further includes a tube extending from the proximal end to said distal end, where said tube is affixed to said male fitting near at or near said proximal end.

12. The male fitting of claim 9, where said first portion includes a rigid thermoplastic or a metal.

13. The male fitting of claim 9, where said second portion has a length from said first portion to said distal end, and where said length is approximately 6 mm.

14. The male fitting of claim 10, where said male fitting, when coupled to a female fitting that conforms to International Standard ISO 594, has a dead space with a volume of less than 15 μl.

15. A male fitting conforming to International Standard ISO 594, where said male fitting is connectable to a female fitting conforming to International Standard ISO 594, and where said male fitting connected to said female fitting has a dead space, where said dead space has a volume of less than 15 μl.

16. The male fitting of claim 15, where said male fitting extends longitudinally from proximal end to a distal end, said male fitting further comprising:
a first portion for mating with a matched female fitting; and
a second portion having a length from said first portion to said distal end, where said length is greater than approximately 1.75 mm.

17. The male fitting of claim 16, where said second portion includes an elastomeric material or a metal.

18. The male fitting of claim 16, where said male fitting further includes a tube extending from the proximal end to said distal end, where said tube is affixed to said male fitting near at or near said proximal end.

19. The male fitting of claim 16, where said first portion includes a rigid thermoplastic or a metal.

20. The male fitting of claim 16, where said length is approximately 6 mm.

21. The male fitting of claim 15, where said male fitting extends longitudinally from proximal end to a distal end, said male fitting further comprising:
a first portion for mating with a matched female fitting; and
a second portion between said first portion and said distal end, where said second portion includes an elastomeric material.

22. The male fitting of claim 21, where said male fitting further includes a tube extending from the proximal end to said distal end, where said tube is affixed to said male fitting near at or near said proximal end.

23. The male fitting of claim 21, where said first portion includes a rigid thermoplastic.

24. The male fitting of claim 21, where said second portion has a length from said first portion to said distal end, and where said length is approximately 6 mm.
